(19) United States
(12) Patent Application Publication
(10) Pub. No.: US 2022/0330961 A1
(43) Pub. Date: Oct. 20, 2022

(54) CLOT RETRIEVER AND THROMBECTOMY DEVICE FOR MASSIVE CLOT REMOVING FROM VASCULAR SYSTEM WITH ADJUSTABLE DEVICE ORIFICE AND RETRIEVE LENGTH

(71) Applicant: Mehmet Hakan Akpinar, Kadiköy/Istanbul (TR)

(72) Inventors: Mehmet Hakan AKPINAR, Kadiköy/Istanbul (TR); Ibrahim Faruk AKTÜRK, Üsküdar/Istanbul (TR); Özlem HORZUM BAYIR, Sancaktepe/?stanbul (TR)

(73) Assignee: Mehmet Hakan Akpinar, Kadiköy/Istanbul (TR)

(21) Appl. No.: 17/720,420

(22) Filed: Apr. 14, 2022

Related U.S. Application Data
(60) Provisional application No. 63/174,647, filed on Apr. 14, 2021.

(51) Int. Cl. A61B 17/221

(52) U.S. Cl. CPC A61B 17/221; A61B 2017/00867

(57) ABSTRACT

The invention relates to a clot retrieval and thrombectomy device with adjustable orifice diameter and retrieval length to retrieve embolism from the vascular system, especially pulmonary arteries and venous system, which allows restoring the blood flow to pulmonary vasculature and relieve right heart strain and re-perfusion in occluded veins or arteries with organized thrombus formation. The main objective of the invention is to provide a device that can be used in different vessel dimensions, different types of vessels and situations. The present invention has an adjustable device orifice and retrieval length to overcome the limitations in retrieval of the clots in different vessel dimensions. According to the clot length and volume, the orifice of the braided mesh basket can be adjusted to the vessel diameter. Therefore, the device can be used in every type of vessel having different diameters and in every patient having different conditions.

### CROSS-REFERENCE TO RELATED APPLICATIONS

The present application claims priority to and the benefit of U.S. Provisional Patent Application No. 63/174,647, filed Apr. 14, 2021, the contents of which are hereby incorporated in their entirety.

### TECHNICAL FIELD

This invention relates to a clot retrieval and thrombectomy device with adjustable orifice diameter and retrieval length to retrieve embolism from the vascular system, especially pulmonary arteries and venous system, which allows restoring the blood flow to pulmonary vasculature and relieve right heart strain and re-perfusion in occluded veins or arteries with organized thrombus formation.

### BACKGROUND

Venous thromboembolic disease is a common condition with high morbidity and mortality. Deep vein thrombosis and pulmonary embolism (PE) affect a large population worldwide. The patients with PE are treated with systemic anticoagulation when any contraindication occurs. Patients with massive PE, defined as acute PE with hemodynamic compromise, are typically treated more aggressively due to high associated morbidity and mortality. It is known that acute pulmonary embolism is one of the leading causes of death, morbidity, and hospitalization. Right ventricular failure is the most common cause of early death in these patients.

The catheter-based thrombectomy may be the only viable option in patients with life-threatening PE who are not suitable candidates for either surgical embolectomy or thrombolysis. Percutaneous mechanical thrombectomy (PMT) is accepted as a treatment in patients with high-risk acute pulmonary embolism with contraindications to systemic thrombolysis or when the treatment has failed.

In the prior art, there are several methods for the treatment of PE. Multiple percutaneous techniques have been developed and used to manage acute massive PE during the last decade. Local catheter-directed thrombolytic (CDT) therapy, thrombus aspiration with mechanical fragmentation techniques, and percutaneous mechanical thrombectomy (PMT) are the established treatment methods for acute massive PE patients. Pharmacomechanical thrombolysis is a practical approach to restore pulmonary circulation, but previous studies have indicated that a significant percentage of PE patients have substantial contraindications to thrombolytic or anticoagulation therapy. PMT should be an option in those cases.

Another treatment method, catheter-directed thrombolysis treatment, is a standard treatment method and it is helpful, especially in thrombolysis, over approximately 24 hours. This approach may not provide hemodynamics rapidly enough to achieve improvement in clinical outcomes in critically ill patients. In these cases, other endovascular techniques, such as clot fragmentation and thrombectomy, may be used. Balloon fragmentation of pulmonary emboli technique which is another treatment method, has been used to provide rapid pulmonary blood flow restoration and promote cardiac output. Also aspiration thrombectomy can be performed manually through a large sheath to increase total pulmonary perfusion and reduce pulmonary artery pressure to provide rapid improvement in unstable PE patients. Recently, new aspiration thrombectomy devices have entered the market with specific PE intervention indications as a large-bore aspiration thrombectomy (LBAT) catheter. This technique offers the potential of a stand-alone treatment for acute massive and submassive PE without the hemorrhagic complication rates associated with the use of CDT.

The percutaneous mechanical thrombectomy (PMT) method is usually performed by manipulation with conventional pigtail catheters and angioplasty balloons, and it has been shown to reverse systemic hypotension, decrease PAP, and improve arterial oxygenation. The success rate of a PMT technique could be defined as the percentage volume of thrombus cleared or fragmented during the treatment into sufficiently small particles.

A problem with the prior art devices is that most PMT devices range in caliber from 5 to 8 F. Smaller devices produce lower vascular trauma and also cause lower bleeding at the access site. On the other hand, smaller catheters usually have less torque control and tend to have lower efficiency, making them less effective in treating large thrombi. In the prior art, there are some devices to overcome the limitations of smaller systems in their ability to entrap large debris en bloc. However, these devices' major limitation is cannula stiffness and trackability into the pulmonary route because of significant vascular injury potential.

One significant limitation of the existing technology is that the orifice of the available devices' capturing system has a constant diameter and length, which can not be adjusted to the target vessel dimensions (width/diameter and length), or the clot size and volume. The clot retrieving and thrombectomy procedure has limitations for the vessel dimensions' variation due to lacking an adjustable device system.

Another limitation of the existing technology is that in the available clot removal devices, firstly a wire must be inserted through the clot or blockage. Then the wire can deploy and expand after passing the clot or blockage, which means that the wire opens in a position ahead of the clot and pull back the clot or blockage. In other words, known devices have a mechanism that obligate to pass through the clot, and after passing through the clot they expand and then take the clot back. However in some cases, there is not enough space in the vessel for the retrieval device to go beyond the clot by inserting the device through the clot. Known devices require a space located beyond the clot, wherein the space must be at least in the size of the clot. Therefore, usage of available devices are limited depending on the location of the clot and dimensions of the vessel. The usage of these devices by passing through and going beyond the clot, can cause disadvantages like limiting the usage area and they cannot be used in all types and sizes of vessels and all types of patients and conditions. Another limitation of the prior art is that the available devices are incapable of retrieving organized and solid/rigid thrombus, and they are mostly used for removing thrombus having loose structure. For the organized and solid/rigid thrombus, extensive surgical procedures are required and rapid action cannot be taken in many cases and they result in mortality generally.

In U.S. Ser. No. 10/722,255 B2, systems and methods for removing obstructive matter from body lumens and treating vascular defects are disclosed. The system described in U.S. Ser. No. 10/722,255 B2 can include an outer shaft having a lumen, sidewall, and a longitudinal window in the sidewall, an inner shaft disposed within the lumen, and an expandable member having a first end coupled to the outer shaft and a second end coupled to the inner shaft through the window. The expandable member can be expanded to an expanded configuration by relative movement of the outer and inner shaft. This system has a technique that the obstructions can be trapped between the outer shaft having a lumen, sidewall, and a longitudinal window in the sidewall. After that, the trapped obstruction is retracted out of the body. However, the device described herein has an expansion process depending on the catheter diameter, and the system does not allow adjusting the device diameter to the variable vessel diameters. The outer diameter of mentioned device depends on the catheter diameter, which limits the maximum expanded diameter. Therefore, the device cannot perform different calibrations of the vessels and is insufficient in removing obstructive matter in all situations.

Another prior art, U.S. Ser. No. 10/292,722 B2, discloses a clot retrieval device for removing a clot from a blood vessel comprising an elongate member or shaft having a proximal end that extends the patient's exterior so that a user can retrieve the stent-basket device and capture the clot by retracting the shaft. This device has a system that needs to be fully expanded after passing through the clot location to start to retrieve. Mentioned device has a fixed diameter and this fixed diameter feature creates a potential gap when expanded in an undersized blood vessel (smaller size than device) which causes a disadvantage of blood flow through the gap. In addition, since the outer diameter has a fixed size and cannot be adjusted to the vessel wall diameter, said device is not sufficient to remove a clot in all situations and in all vessels.

EP 3579765 A1 discloses systems and methods which can remove material of interest, including blood clots, from a body region, including but not limited to the circulatory system for the treatment of pulmonary embolism (PE), deep vein thrombosis (DVT), cerebrovascular embolism, and other vascular occlusions. The system allows a compressed reserve segment to be stored along length of a catheter system in a compact manner, thus providing a long effective capture length tubular mesh without requiring an entire system to include a long fixed length. However, said stent cannot be fixed to a certain diameter when it is expanded so the device does not have a control mechanism to prevent the damage to the surrounding tissue during usage.

According to the problems in the prior art, such as constant diameter of the orifice of the available devices' capturing systems, which can not be adjusted according to the target vessel dimensions or the clot's size and volume, incapability of retrieving organized and solid/rigid thrombus, requirement of a space located beyond the clot for retrieval, wherein the space must be at least in the size of the clot, limited usage in different vessel dimensions, variations and insufficiency to remove a clot in all situations; a clot retrieval and thrombectomy device that overcomes these problems is needed in this technical field.

### SUMMARY

The present invention discloses a device that provides retrieving for embolism from the vascular system, especially pulmonary arteries, and venous system. The present invention is a percutaneous clot retriever and mechanical thrombectomy device which is used to retrieve acute and massive PE through a combined metallic mesh device with a gentle outward radial force. The combined metallic mesh provides entrapping the thrombus, often dragging large pieces of thrombotic debris back into the guide catheter, and concomitant thrombolysis treatment can be performed simultaneously.

The main objective of the invention is to provide a device that can be used in different vessel dimensions, different types of vessels and situations. The present invention has an adjustable device orifice and retrieval length to overcome the limitations in retrieval of the clots in different vessel dimensions. According to the clot length and volume, the orifice of the braided mesh basket can be adjusted to the vessel diameter. Therefore, the device can be used in every type of vessel having different diameters and in every patient having different conditions. In the prior art the orifice is fixed to the catheter which limits the adjustment (both enlargement and reduction in orifice diameter).

In some cases, there is not enough space for the retrieval device to go beyond the clot by inserting the device through the clot. Devices in the prior art require a space located beyond the clot, wherein the space must be at least in the size of the clot. Therefore, usage of available devices are limited depending on the location of the clot and dimensions of the vessel. However, the present invention do not require a space that at least in the size of the clot, located beyond the clot. The invention grabs and traps the clot even the vessel do not contain a space located beyond the clot. The invention comprises a guidewire that passes through the clot like other available devices; however after passing the clot, it does not need to pass at least as long as the clot length, the guidewire only passes through the clot until the end of the clot length.

The orifice of the device can be adjusted (enlarged or reduced/narrowed) to the intended diameter, in contrast to the prior art, and the braided mesh basket can be extended according to the clot length and volume precisely by replacement of three catheters, including a delivery sheath, outer shaft, an inner shaft, respectively, which correlates with diameter variation by the help of the handle gear and locking mechanism. The outer diameter of the present invention depends on the self-expandable stent expansion. Thus, the diameter of the orifice and the retrieval length can be adjusted according to the vessel diameter precisely and provides more extensive and safe usage. Moreover, the braided mesh basket's expanded length can be adjusted precisely to retrieve the clot inside the delivery catheter.

Another objective of the invention is to provide a device that can be easily applied by doctors who can use the transcatheter method. In the prior art, the available devices are incapable of retrieving organized and solid/rigid thrombus in fatal situations, and they are mostly used for removing thrombus having loose structure. For the organized and solid/rigid thrombus, extensive surgical procedures are required and rapid action cannot be taken in many cases and they result in death of patients generally.

Another objective of the invention is to ensure that clot cannot migrate or embolize to the distal part of the lumen. The invention traps the clot or obstruction inside the braided mesh basket by reducing the diameter of the stent, ensuring the clot cannot migrate or embolize to the distal part of the lumen.

Another objective of the invention is to prevent damage to the surrounding tissue during procedure. Expanded diameter of the orifice of the invention device can be adjusted precisely to the vessel wall dimension considering outcomes of the CT, fluoroscopy, TEE, TTE, ICE, etc. by fixation feature of the handle to prevent damage to the surrounding tissue and vessel wall. Also, the orifice of the device can be locked to safely retrieve the thrombus/clot inside the catheter.

The present invention provides a device;

with adjustable orifice diameter which can be both enlarged and reduced according to the vessel and clot dimensions to be used in all situations, in different blood vessel dimensions and variations,

that is capable of retrieving organized and solid/rigid thrombus,

that does not require a space at least in the size of the clot, located beyond the clot for retrieval,

with an adjustable retrieval length according to the clot length,

a locking mechanism to fix the orifice diameter and retrieval length to safely remove the clot.

### BRIEF DESCRIPTION OF THE DRAWINGS

FIG. 1 is a schematic illustration of the device showing a first element (103) that is retracted into an outer sheath (101).

FIG. 2 is a schematic illustration of the device showing the first element (103) that is partially expanded from the outer sheath (101).

FIG. 3 is a schematic illustration of the device showing the first element (103) that is expanded from the outer sheath (101) entirely.

FIG. 4 is a schematic illustration of the device showing a second element (106) expanding from the inside of the first element (103) that is passing from the outer sheath (101).

FIG. 5 is a schematic illustration of the device showing the second element (106) expanding from the inside of the first element (103) partially, wherein the second element (106) is connected to the first element (103) by one or more connection wires (107) resulting in a third body.

FIG. 6 is a schematic illustration of the device showing the second element (106) expanding from the inside of the first element (103) entirely, passing from the outer sheath (101) through a guidewire lumen (104) which is connected to the second element (106) comprising a braided mesh with a welding ring (108) welded from one side of the internal shaft (102) and from one side of the second element (106).

FIG. 7 is a schematic illustration of the device showing the first element (103) that is retracted into the external shaft (105) inside the outer sheath (101) through a guidewire lumen (104) partially.

FIG. 8 is a schematic illustration of the device showing the adjustable orifice's cross-sections through pushing or retracting the external shaft (105) (A. the smallest orifice diameter when the device is fully retracted by external shaft (105), B. the orifice diameter as semi-opened configuration when the device is pushed/advanced by external shaft (105) partially, C. the orifice diameter as a semi-opened configuration when the device is pushed until the end of the external shaft (105), D. the largest orifice diameter as a fully expanded configuration when the device is pushed out of the external shaft (105) entirely).

FIG. 9 is a schematic illustration of the device showing all the main components of the catheter.

FIG. 10 is a schematic illustration of the device showing a removable handle body (116) comprising a first locking mechanism (110), a second locking mechanism (111), a rotating rack, and pinion progress lever (114).

FIG. 11 is a schematic illustration of the device showing all the main components of the device with catheter.

### DESCRIPTION OF THE REFERENCES

101. outer sheath
102. internal shaft
103. first element
104. guidewire lumen
105. external shaft
106. second element
107. connection wire
108. welding ring
109. radiopaque tip
110. first locking mechanism
111. second locking mechanism
112. a side port for contrast media injection
113. catheter handle
114. rotating rack and pinion progress lever
115. guidewire
116. handle body

### DETAILED DESCRIPTION OF ILLUSTRATIVE EMBODIMENTS

The present invention discloses a clot retrieval and thrombectomy device with adjustable orifice diameter and retrieval length for massive clot removal to retrieve embolism from the vascular system, especially pulmonary arteries, and venous system.

The disclosure for a preferred embodiment is described with the related drawings and references. This disclosure may be embodied in different designs and shapes and should not be limited to the archetype. Specific embodiments are described to complete the disclosure scope for those skilled in the art. However, the wording used to describe the disclosure and the related drawings in detailed explanations are not a limitation for the similar descriptions.

The present invention comprises;
a first element (103) comprising a stent-like metallic structure having at least one shape memory metallic strut made of expandable medical grade metallic material, wherein the orifice diameter of the first element (103) is adjustable, wherein the orifice diameter of the first element (103) is enlarged and reduced according to the vessel and clot dimensions,
a second element (106) comprising a reverse braided mesh basket having at least one wire made of expandable medical grade metallic material, which is connected to the first element (103),
a welding ring (108) for holding the ends of the reverse braided mesh basket of the second element (106) together,
an external shaft (105) that is partially or entirely made of a metallic tubing, which is connected to the first element (103) and welded to the second element (106) through the welding ring (108),
an internal shaft (102) made of a metallic tubing with a specific calibration, which comprises a guidewire lumen (104) to navigate the device through the clot,
an outer sheath (101) covering the external shaft (105) and the internal shaft (102), wherein the orifice diameter of the first element (103) is enlarged and reduced by changing the tip location of the external shaft (105) according to the vessel wall diameter,
connection wire (107) made of at least one shape memory metallic material, wherein second element (106) is connected to the first element (103) by at least one connection wire (107),
a rotating rack and pinion progress lever (114) to adjust the orifice diameter of the device by rotating clockwise,
a first locking mechanism (110) to fix the external shaft (105) to the outer sheath (101) after the orifice of the device is adjusted to the vessel diameter,
a second locking mechanism (111) to fix the internal shaft (102) to the outer sheath (101) after the length of the device is adjusted to the clot length,
a handle body (116) either removable or irremovable for precise calibration of the orifice diameter and length.

Herein “orifice” is written as the orifice of the device, however actually it is the orifice of the first element (103). Since the present invention device comprises the first element (103), the orifice is also the device's orifice and named as “orifice of the device” in the description generally. In other words, the orifice diameter of the first element (103) and the orifice diameter of the device is equal. Herein, “welded” is used as “attached”, therefore any attachment technique can be used instead of welding.

The device comprises an outer sheath (101), an internal shaft (102), a first element (103) constructed by a stent-like frame, a guidewire lumen (104) to push and retrieve the guidewire (115), an external shaft (105) connected to the first element (103), a second element (106) constructed by a braided mesh and connected to the first element (103) of the device by at least one connection wire (107) resulting in a third body. Third body comprises the first element (103) and the second element (106) that are connected by at least one or more connection wires (107). The device also comprises a radiopaque tip (109) to provide clarity to the doctor in terms of visualization and to indicate the suitable length of the device that passes through until the clot length.

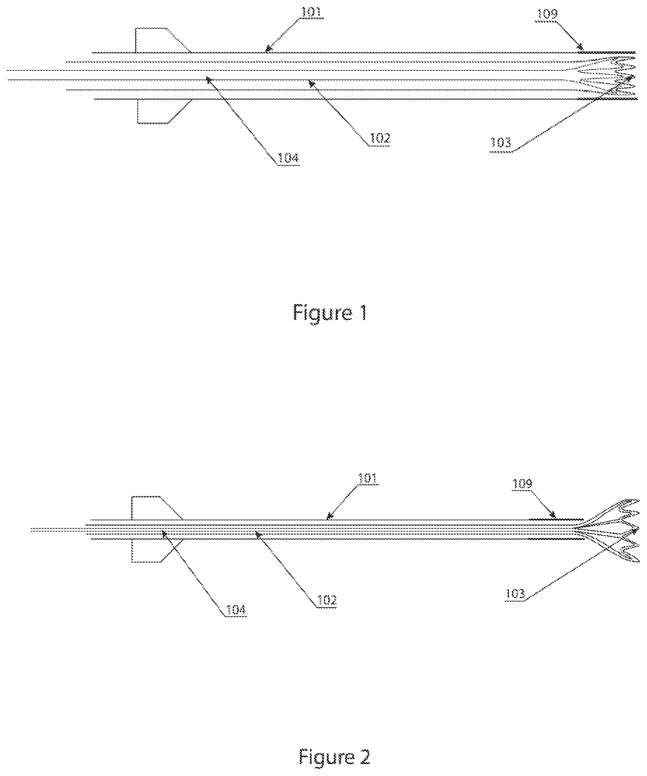

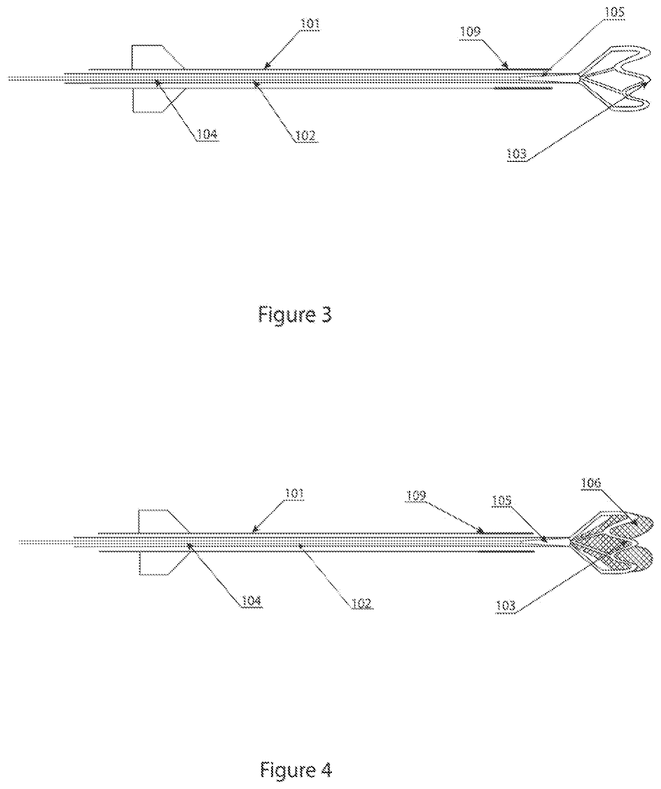

In FIG. 1, the first element (103) is constructed by a stent-like frame which is retracted into the outer sheath (101) partially. In FIG. 2, the first element (103) is partially expanded from the outer sheath (101) and in FIG. 3, the first element (103) is expanded from the outer sheath (101) entirely. It can be seen from the FIG. 4 that the device comprises a second element (106) constructed by a braided mesh, which is expanding from the inside of the first element (103) that is passing from the outer sheath (101).

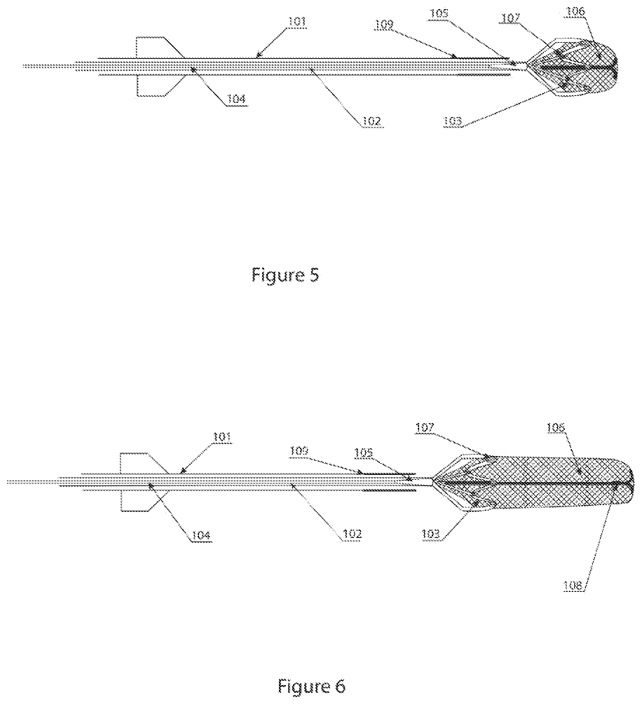

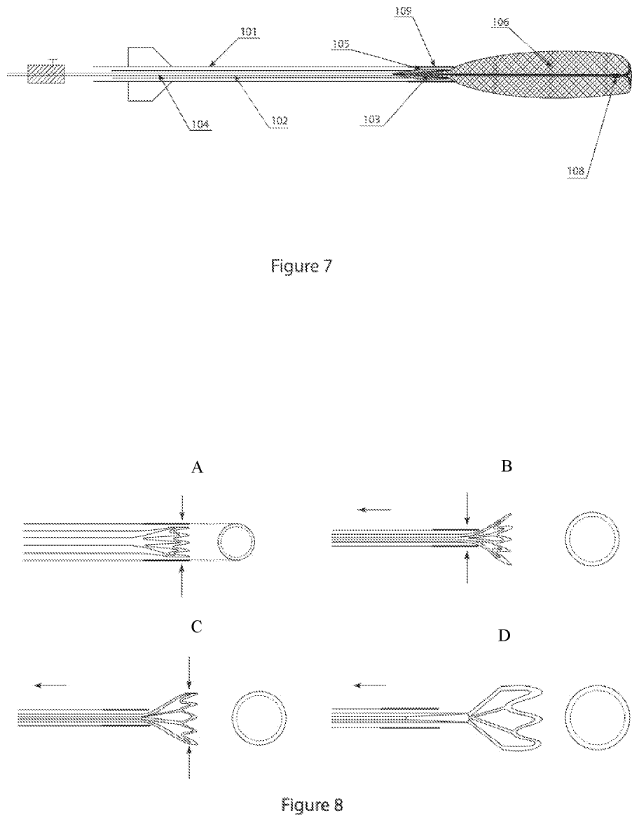

In FIG. 5, the second element (106) is expanding from the inside of the first element (103) partially. In FIG. 6, the second element (106) is expanding from the inside of the first element (103) entirely and in FIG. 7, the first element (103) is retracted back partially into the external shaft (105) located inside the outer sheath (101) through a guidewire lumen (104).

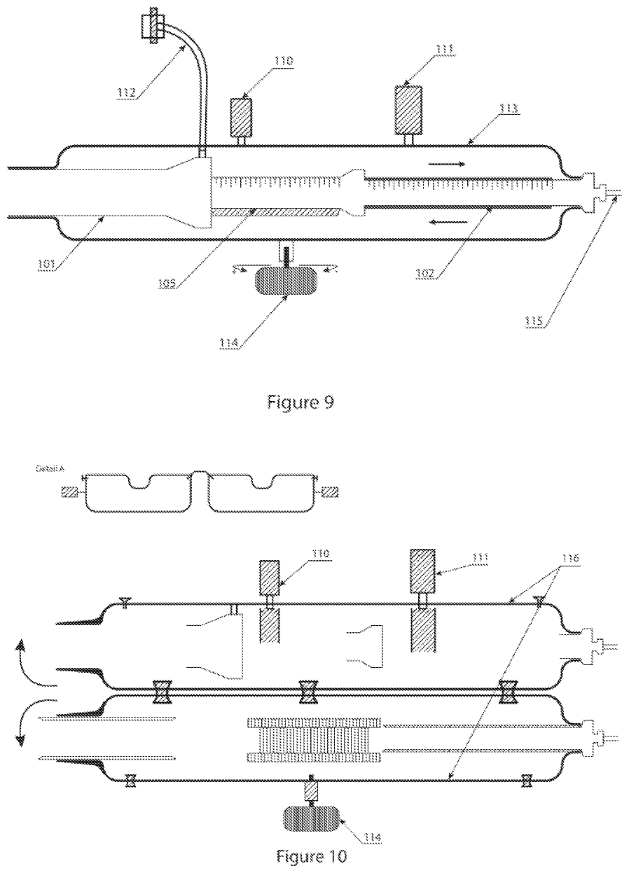

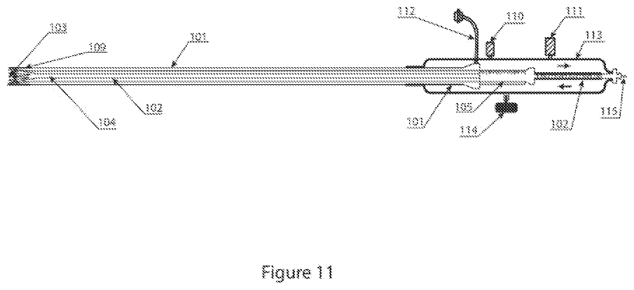

In some of the embodiments of the invention, the device comprise a guidewire (115) that is configured to be placed into the vascular system passing through the clot location after an introducer system is put to the vascular access through the transfemoral approach (FIG. 9, 10, 11). In FIG. 9, all the main components of the catheter are shown. In FIG. 10, a removable handle body (116) comprising a first locking mechanism (110), a second locking mechanism (111), a rotating rack, and pinion progress lever (114) is shown. In FIG. 11 all the main components of the device with catheter is shown, including an outer sheath (101), an internal shaft (102), a first element (103), a guidewire lumen (104), an external shaft (105), a radiopaque tip (109), first locking mechanism (110), a second locking mechanism (111), a side port for contrast media injection (112), the main body of the catheter handle (113), a rotating rack and pinion progress lever (114) and a guidewire (115). In this embodiment, the device is loaded to a loader and flushed with a saline solution to eliminate the risk of residual air bubbles, which might cause air embolization. The third body is configured to be pushed over a guidewire (115) previously crossed the clot location and properly placed on the target vessel's distal section.

The third body can be oriented along the guidewire (115) and pushed/advanced over the target clot location. The third body is expanded when the target clot location is passed until the end of the clot. When the target location is determined under fluoroscopy, the contrast media is injected into the side port for contrast media injection (112), and the radiopaque tip (109) of the catheter is located in the target location. The stent-like first element (103) is started to expand by the rotating rack and pinion progress lever (114) on the main body of the catheter handle (113), by rotating the rotating rack and pinion progress lever (114) in clockwise direction until it reaches the surrounding vessel diameter. The external shaft (105) is fixed at the determined location by the first locking mechanism (110). Then, the catheter system is pulled back precisely until the orifice reaches the clot's external surface and the vessel wall's internal surface. The internal shaft (102) is pushed through the device to create sufficient volume to retrieve the entire clot. The internal shaft (102) is fixed by a second locking mechanism (111) to keep the existing dimensions of the braided mesh second element (106).

The first element (103) is released from the external sheath (101) and captures the clot, and the device is pushed over the guidewire (115) until the device passes the clot location with a suitable length that just passes the clot (until the end of the clot) is visualized with a radiopaque tip (109) on the external sheath (101). The outer sheath (101) of the device is pulled back to release the second element (106) to expand to the target diameter by adjusting the drawn back length of the external sheath (101) calibrated by the dimensions of the second element (106) structure.

The device's adjustable orifice is shown in FIG. 8 which indicates the adjustable orifice's cross-sections that are adjusted through pushing or retracting the external shaft (105) is shown. It can be seen from FIG. 8 that when the external shaft (105) is pushed, the diameter of the orifice enlarges. The further the external shaft (105) is pushed, the wider the orifice gets. The outer sheath (101) is pulled back until the orifice of the first element (103) reaches the vessel wall to cover all the desired location cross-section, avoiding any residual area where the clot can embolize distally create an occlusion in distal vessels when the orifice reaches the desired diameter where the distal part of the clot is located. The outer sheath (101) is locked over the internal shaft (102) and starts to pull back the device gently to trap the clot inside the third body. Meanwhile the amount of clot that is trapped is increasing the volume inside the third body which is increased respectively by pushing the internal shaft (102). When the total clot is entrapped inside of the third body by pulling the entire device proximally, the outer sheath (101) is pushed over the external shaft (105) until the first element (103) totally goes inside the external sheath (101) to close the orifice of the second element (106) to avoid any particles from the clot formation embolized while retrieving the device from the location of the intervention. When the orifice is closed and secured, the total device will be retrieved until the tip of the introducer sheath.

In some embodiments of the invention, the clot retriever and thrombectomy device comprises a first element (103) and a second element (106) and a third body comprising the said first element (103) and the second element (106), which are connected by at least one or more connection wires (107). The device also comprises an internal shaft (102), having a proximal end connected to the first element (103) of the device to entrap, capture and retrieve a thrombus by retracting the outer sheath (101) extending on the internal shaft (102). The second element (106) functions as a mesh sack and presents a more expandable structure than the first element (103) to a radial extent in the deployed configuration to conform to the artery or vein anatomy and capture the thrombus by entrapping. The first element (103) provides a reception area to retract the captured clot to the outer sheath (101).

One embodiment of the invention comprises an outer sheath (101) made of a polymeric tubing reinforced with braided wires or coils having an internal polytetrafluoroethylene (PTFE) liner for a lower coefficient of friction with a hub having a hemostatic valve and a side port for contrast media injection (112) and also an atraumatic radiopaque tip (109).

One embodiment of the invention comprises an internal shaft (102) made of metallic tubing with a specific calibration to be used as a guidewire lumen (104) to navigate the entire device.

One embodiment of the invention comprises an internal shaft (102) made of a metallic tubing with a specific calibration to be used as a guidewire lumen (104) to navigate the entire device, which is connected to the second element (106) comprising a braided mesh with a welding ring (108) welded from one side to the internal shaft (102) and from the other side to the second element (106).

One embodiment of the invention comprises an external shaft (105) partially or entirely made of a metallic tubing with a specific calibration to be used as an internal shaft (102) lumen to be welded first element (103) by changing the tip location of the external shaft (105) concerning outer sheath (101) designed to increase and decrease the orifice of the stent-like first element (103) to adjust the diameter according to the vessel wall dimensions.

One embodiment of the invention comprises a stent-like first element (103) having one or more struts made of a shape memory, expandable metallic material such as nitinol or other medical grade metallic materials.

In one embodiment of the invention, the first element (103) is constructed by laser-cut, braiding, or combined methods.

In some embodiments of the invention, the device comprise a second element (106), having one or more wires made of shape memory, expandable metallic materials such as nitinol or other medical grade materials. The second element (106) is constructed by laser-cut, braiding, or combined methods. The second element (106) comprises a welding ring (108) which holds the wires together to create a mesh structure and a connection point to the internal shaft (102), which allows an internal lumen to be used as an extension of the guidewire lumen (104).

In one embodiment of the invention, the first element (103) is connected to the second element (106) with one or more connection wires (107) obtained by passing metallic wires, one of the components of the second element (106), through the strut gaps of the first element (103) or one or more connection wires (107) are used to connect the first element (103) to the second element (106).

One embodiment of the invention comprises one or more connection wires (107) made of a shape memory metallic material such as nitinol or similar metallic materials to hold the first element (103) and the second element (106) together, passing metallic wires of the second element (106) from the strut gaps of the first element (103).

One embodiment of the invention comprises a third body including at least a first element (103) and a second element (106) which has a welding ring (108) to hold the wires together and create a connection point to the internal shaft (102) to push and retract the third body into the external sheath (101). The internal shaft (102) comprises a guidewire lumen (104) to navigate the device through the clot to the target location.

In one embodiment of the invention, the welding ring (108) made of a metallic material such as 304 stainless steel or similar materials, is pushed into the first element (103) lumen so that the metallic mesh structure of the second element (106) pulled inward is pushed up to the external shaft (105) to which the first element (103) is connected, and creates a connection point for the internal shaft (102) in the outer sheath (101).

In one embodiment of the invention, the device comprises a variable orifice of the second element (106) that can be adjusted to the surrounding vessel wall diameter avoiding a gap between the vessel wall and device orifice for a potential cause the distal embolization. The second element (106) does not require a long distance in the vascular system distal side of the clot location, whereas the needed capturing volume can be provided by feeding the second element (106) through the guidewire lumen (104) as long as possible to maintain a sufficient capturing volume to retrieve the entire clot.

In one embodiment of the invention, the second element (106) can be elongated with additional feeding from the internal shaft (102) lumen to obtain the same volume of the entrapped clot with a longer length but smaller diameter to decrease the clot diameter to fit the smaller calibration vessels during the retrieval.

One embodiment of the invention comprises a removable handle body (116) constructed by two pieces of handle cover providing the feeding of the outer sheath (101), external shaft (105), and internal shaft (102) individually to improve the flexibility of the device. The removable handle body (116) fixes the outer sheath (101), external shaft (105), and internal shaft (102) fed into the body, closing its covers. The removable handle body (116) fixes the outer sheath (101), external shaft (105), and internal shaft (102) at the desired location and for the precise adjustment of the orifice with the rotating rack and pinion progress lever (114).

What is claimed:

1. A clot retrieval and thrombectomy device for massive clot removing from the vascular system with adjustable device orifice and retrieve length characterized by comprising:
an external shaft (105) partially or entirely made of a metallic tubing welded to the second element (106) through a welding ring (108),
an internal shaft (102) made of a metallic tubing with a specific calibration,
an outer sheath (101) extending on an external shaft (105) and internal shaft (102),
a first element (103) comprising a stent-like metallic structure having at least one or plural shape memory metallic struts made of nitinol or similar medical grade metallic materials,
a second element (106) comprising a reverse braided mesh having at least one or plural wires made of nitinol or similar medical grade metallic materials,
connection wires (107) consisting of one or plural shape memory metallic materials such as nitinol or similar metallic materials,
a welding ring (108) to hold the end of the braided mesh's metallic wires together,
a rotating rack and pinion progress lever (114) to adjust the orifice of the device system rotating clockwise,
a first locking mechanism to fix the external shaft (105) to the outer sheath (101),
a second locking mechanism to fix the internal shaft (102) to the outer sheath (101), and
a handle body either removable or irremovable for precise calibration of the orifice diameter and length.

2. A clot retrieval and thrombectomy device according to claim 1, characterized by comprising:
a first element (103) comprising a stent-like metallic structure having at least one shape memory metallic strut made of expandable medical grade metallic material, wherein the orifice diameter of the first element (103) is adjustable, wherein the orifice diameter of the first element (103) is enlarged and reduced according to the vessel and clot dimensions,
a second element (106) comprising a reverse braided mesh basket having at least one wire made of expandable medical grade metallic material, which is connected to the first element (103),
a welding ring (108) for holding the ends of the reverse braided mesh basket of the second element (106) together,
an external shaft (105) that is partially or entirely made of a metallic tubing, which is connected to the first element (103) and welded to the second element (106) through the welding ring (108),
an internal shaft (102) made of a metallic tubing with a specific calibration, which comprises a guidewire lumen (104) to navigate the device through the clot,
an outer sheath (101) covering the external shaft (105) and the internal shaft (102), wherein the orifice diameter of the first element (103) is enlarged and reduced by changing the tip location of the external shaft (105) according to the vessel wall diameter,
connection wire (107) made of at least one shape memory metallic material, wherein second element (106) is connected to the first element (103) by at least one connection wire (107),
a rotating rack and pinion progress lever (114) to adjust the orifice diameter of the device by rotating clockwise,
a first locking mechanism (110) to fix the external shaft (105) to the outer sheath (101) after the orifice of the device is adjusted to the vessel diameter,
a second locking mechanism (111) to fix the internal shaft (102) to the outer sheath (101) after the length of the device is adjusted to the clot length,
a removable or irremovable handle body (116) comprising the first locking mechanism (110), the second locking mechanism (111), the rotating rack and pinion progress lever (114); wherein the removable handle body (116), fixes the outer sheath (101), external shaft (105), and internal shaft (102) at the desired location for the precise adjustment of the orifice diameter and length with the rotating rack and pinion progress lever (114).

3. The clot retrieval and thrombectomy device according to claim 1, characterized by comprising a radiopaque tip (109) on the external sheath (101) to provide clarity to the doctor in terms of visualization and to indicate the suitable length of the device that passes through until the clot length.

4. The clot retrieval and thrombectomy device according to claim 2, characterized by comprising a radiopaque tip (109) on the external sheath (101) to provide clarity to the doctor in terms of visualization and to indicate the suitable length of the device that passes through until the clot length.

5. The clot retrieval and thrombectomy device according to claim 1, characterized by comprising a side port for contrast media injection (112).

6. The clot retrieval and thrombectomy device according to claim 2, characterized by comprising a side port for contrast media injection (112).

7. The clot retrieval and thrombectomy device according to claim 1, characterized by comprising a guidewire (115) that is configured to be placed into the vascular system for passing through the clot location after an introducer system is put to the vascular access through the transfemoral approach.

8. The clot retrieval and thrombectomy device according to claim 2, characterized by comprising a guidewire (115) that is configured to be placed into the vascular system for passing through the clot location after an introducer system is put to the vascular access through the transfemoral approach.

9. The clot retrieval and thrombectomy device according to claim 1, wherein the outer sheath (101) is made of a polymeric tubing reinforced with braided wires or coils having an internal polytetrafluoroethylene (PTFE) liner.

10. The clot retrieval and thrombectomy device according to claim 1, wherein the expandable medical grade metallic material is nitinol.

11. The clot retrieval and thrombectomy device according to claim 1, wherein the first element (103) or the second element (106) is constructed by laser-cut or braiding or both laser-cut and braiding.

12. The clot retrieval and thrombectomy device according to claim 1, wherein the welding ring (108) is made of a metallic material.
